(19) United States
(12) Patent Application Publication
(10) Pub. No.: US 2007/0112415 A1
(43) Pub. Date: May 17, 2007

(54) Braided stent

(75) Inventor: Jeremy Dennis Bartlett, Surrey (GB)

Correspondence Address: SUGHRUE MION, PLLC, 2100 PENNSYLVANIA AVENUE, N.W., SUITE 800, WASHINGTON, DC, 20037, US

(73) Assignee: ABBOTT LABORATORIES

(21) Appl. No.: 11/652,559

(22) Filed: Jan. 12, 2007

Related U.S. Application Data
(63) Continuation of application No. 10/031,064, filed on Apr. 12, 2002.

(30) Foreign Application Priority Data
Jul. 16, 1999 (GB) 9916812.2
Jun. 1, 2000 (GB) 0013362.9

(51) Int. Cl. A61F 2/06

(52) U.S. Cl. 623/1.15

(57) ABSTRACT

A radially self-expanding stent for implantation in a body passage comprises first and second sets of mutually counter-rotating metallic filaments which are braided together and define a tubular stent body having two ends which is mechanically biassed towards a first radially expanded configuration in which it is unconstrained by externally applied forces and can be retained in a second radially compressed configuration, and in which some or all of the filaments ends at the ends of the body are fixed together in pairs each consisting of counter-rotating filaments by placing the filaments over one another and placing them adjacent to and substantially parallel to one another and further comprising a join at each end fixing to retain the ends of the filaments in contact with one another.

This is a continuation of application Ser. No. 10/031,064 filed Apr. 12, 2002, which is a national stage application under 35 U.S.C. §371 of PCT/GB00/02735, filed Jul. 17, 2000, and which claims benefit of priority based on United Kingdom Patent Application No. 9916812.2, filed Jul. 16, 1999 and United Kingdom Patent Application No. 0013362.9, filed Jun. 1, 2000. The entire disclosures of the prior applications, application Ser. No. 10/031,064, as well PCT/GB00/02735, U.K. Application No. 9916812.2, and U.K. Application No. 0013362.9, are hereby incorporated by reference.

The present invention relates to an implantable stent for transluminal implantation in a body lumen, especially found in peripheral and coronary blood vessels, but also for use in the colon, bile ducts, urethras or ileums.

There are several designs of stents, permanently implantable devices, for transluminal insertion into blood vessels and other lumen to prevent or reverse occlusion or stenosis thereof. There are three basic categories of device, namely heat-expandable devices, balloon-expandable devices and self-expanding devices. The present invention is concerned with self-expanding devices with an optional heat expanding capability, that is which are inserted into the body lumen in a radially compressed condition and which are mechanically biased towards a radially expanded position. Upon being released in the blood vessel at the desired position, the stent expands radially exerting outwardly directed pressure upon the inner surface of the wall of the body lumen in which it is positioned.

One such expanding device which is commercially available is the so-called Wallstent. The device is described in WO-A-83/03752. It consists of two sets of counter-rotating helical filaments of metallic wire which are braided together in a one over/one under pattern.

A difficulty with braided stents in general is the tendency of the filaments at the end of the stent to unravel and splay outwards before or after deployment. This tendency makes the stent difficult to handle and the splayed ends can damage the inside wall of the body vessel in which the stent is deployed. In WO-A-83/03752, it is suggested that the filaments may be joined to one another at the end of the stent. However, as explained in a later specification by Wallsten et al in U.S. Pat. No. 5,061,275, for stents with a high axial braid angle a between counter-rotating filaments, that this rigidifies the ends of the prosthesis and can create unwanted permanent plastic deformation at the joins when stent diameter is changed. This makes it difficult for the stent to freely and reversibly adopt differing diameters.

A new radially self-expanding stent according to a first aspect of the invention adapted for implantation in a body passage comprises first and second sets of mutually counter-rotating metallic filaments which are braided together and define a tubular stent body having two ends which is mechanically biased towards a first radially expanded configuration in which it is unconstrained by externally applied forces and can be retained in a second radially compressed configuration, and in which some or all of the filaments at the ends of the body are fixed together in pairs each consisting of counter-rotating filaments by placing the filament ends over one another and placing them adjacent to and substantially parallel to one another and further comprising a join at each end fixing to retain the ends of the filaments in contact with one another.

A stent with this configuration allows its ends to deform elastically during compression and expansion. The stress created during this process is redistributed over the section of the braid that is adjacent to a joined end and this deforms in a generally elastic manner. Because of this the join has a reduced stress load on it and can recover elastically.

In this case the respective filaments may be shaped such that the ends bend outward radially, and may be configured such that the angle at which they bend outward radially increases towards the end.

The filaments may be folded over one another or partially unfolded at the ends. The fixed ends may be shaped or heat treated to urge the respective filaments to a position in which they are biased out of parallel alignment with the adjacent filament to which they are connected at the region of the join.

Although the welding can be by resistance welding and/or by pressure, it is preferred for heat to be used, generally by spot, laser, or plasma welding. Preferably the welding softens the metal such that it forms a globule before resolidifying to form a bead.

For some embodiments and applications it may be adequate to join some but not all of the filament ends. For instance it may be convenient to weld every third pair of counter-rotating filaments at the end of one or both ends of the stent body. Preferably at least every other pair is welded at both ends, more preferably every pair is welded at one, or preferably both, ends. In any of these cases each filament and may be joined to one of its next-but-one neighbours.

Preferably no filler wire is used in the welding although it may, for some purposes, be useful to include filler wire, for instance where the filler has different, usually greater, radiopacity than the material from which the metal filaments are made. The formation of a bead and/or the use of high radiopacity filler material at the join enables the ends of the stent to be made more radiopaque (to X-rays transmitted perpendicular to the axis) than the body of the stent between the ends. This assists in visualization of the stent during an operation.

If a bead is formed it generally may have a diameter of at least 1.2 times that of the diameter of the filament, for instance at least 1.5 times or as much as or more than 2 times the diameter. The diameter of the bead is usually no more than 3, preferably less than 2.5, times the diameter of the filament. We have found that it assists retention of the stent on a delivery device and its delivery from that device if the bead's periphery extends outwardly beyond the periphery of the stent as defined by the filament surfaces, preferably on the inner wall. This results in the bead providing shoulders on either or both the inner and outer walls which can provide a radially directed surface against which a corresponding radially directed surface on a movable component of a delivery device can bear to impose motion of the stent relative to other components of the delivery device. Preferably each bead provides a shoulder in a rearward (with respect to delivery) axial direction. The shape of the resolidified bead at least on the outer wall of the stent is generally rounded, for instance approximately elliptical, and this provides a smooth external stent surface to minimize damage to the inside wall of the vessel in which the stent is implanted and/or the delivery system in which the stent is placed prior to deployment.

A smooth inner weld surface is also preferable to ensure that the stent does not damage any device on which it is retained or any other mechanical device that may have to pass through it.

It is suitable for heat treatment to be conducted by subjecting the stent either before or after the welding operation to elevated temperatures to harden the metal. For Elgiloy, (available from Fort Wayne Metals) for instance, heat treatment, optionally in a vacuum or inert atmosphere, may be carried out at a temperature in the range 510 to 530° C., for instance around 520° C. for a period of at least 2 hours, preferably about 3 hours.

The first radially expanded diameter is the diameter adopted by the stent when no externally directed force is exerted upon it, that is when it expands freely in air. This diameter is somewhat greater than the internal diameter of the lumen into which stent is to be implanted since this results in the stent exerting a continuous outwardly directed force on the internal wall of the body lumen in which it is located. In this fully unloaded conformation it is preferable that the angle a between filaments is less than 90°. Preferably in the range 70-89°, most preferably in the range 80° to 89°.

Preferably the mutual angle at which the filaments are fixed is in the range 0° to 15°.

The metallic stent is generally provided with a biocompatible coating, in order to minimize adverse interaction with the walls of the body vessel and/or with the liquid, usually blood, flowing through the vessel. The coating may also allow delivery of a drug. The coating is preferably a polymeric material, which is generally provided by applying to the stent a solution or dispersion of preformed polymer in a solvent and removing the solvent. Non-polymeric coating materials may alternatively be used. Suitable coating materials, for instance polymers, may be polytetrafluoroethylene or silicone rubbers, or polyurethanes which are known to be biocompatible. Preferably however the polymer has zwitterionic pendant groups, generally ammonium phosphate ester groups, for instance phosphoryl choline groups or analogues thereof. Examples of suitable polymers are described in our earlier application number WO-A-93/01221. Particularly suitable polymers described in that specification are those which are cross-linkable after coating, since these remain stably adhered to the surface. We have described other suitable biocompatible coating polymers which may be used in WO-A-98/30615. Polymers as described in those specifications are hemo-compatible as well as generally biocompatible and, in addition, may be lubricious.

It is important to, ensure that the metallic surfaces of the stent are completely coated in order to minimize unfavorable interactions, for instance with blood, which might lead to thrombosis in cases where this is not desirable. Although it may be possible to avoid the exposure to blood or metal surfaces at the crossover points, on the mutually contacting portions of the filaments, by sheathing the entire crossover points and hence fixing the filament to one another, as described in DE-A-4240177, it is preferred that the crossover points along the body of the stent should not be fixed to one another but should be allowed to move, for instance to slide or rotate relative to one another. It is thus preferred for the coating to cover entirely the wires even at the crossover points. This can be achieved by suitable selection of coating conditions, such as coating solution viscosity, coating technique and/or solvent removal step.

It is preferred that each filament of the stent should execute at least one full turn of the helix. If the filaments execute less than a full turn, even with the joining of the ends of the filaments, the stent may be relatively unstable. Preferably each filament executes at least 6 turns, though generally less than 12 turns. It is preferred that the stent be formed from at least 4, more preferably at least 8 and most preferably at least 12 filaments in each direction. The number of filaments depends at least in part upon the diameter of each filament as well as the desired diameter and the desired size of the openings between the filaments of the stent in its radially expanded and contracted condition. The number of filaments and their diameter affects the flexibility of the stent in its radially contracted condition during delivery. Generally the number of filaments in each direction is 32 or less and more preferably from 24 downwards.

The filaments may be made from circular section wire. It may, alternatively be advantageous for rectangular section wire to be used, for instance as described in DE-A-4240177 and in the early Wallsten patent WO-A-83/03752. The use of flat (rectangular section wire) may provide optimum radial strength characteristics whilst minimising the overall thickness of the stent, especially at the crossover points, thereby minimising any interference of the liquid flow in the body passageway. The area of contact between wires at the crossover points can be maximized, if required, by the use of flat wire which increases the amount of friction between the wires upon relative movement, for instance during any changes in radius. This should increase the resistance of the expanded stent to radial contraction in use. The use of oval wire (with the smaller dimension being arranged substantially radially with respect to the stent axis) may provide a particularly advantageous combination of strength whilst minimising the contact area at crossover points.

The braiding is usually in a one over-one under pattern although other patterns such as one under-two over or two under-two over could be used.

The thickness of the filaments depends upon the desired final diameter (open diameter) of the stent. Wire having a diameter of 0.04 mm upwards, for instance up to 0.20 mm may be used. Wire with diameters at the lower end of the range would generally be used for making stents for use in small blood vessels, for instance in coronary arteries, where the diameters of the stents is generally in the range 0.5 mm up to 4.0 mm (fully radially expanded diameter). Larger stents may be used in peripheral blood vessels, aortic aneurisms or in stents for use in urological passageways, the oesophagus and in the bile duct, where the stent may have a diameter up to about 30 mm.

The length of the stent in the fully unloaded conformation maybe in the range 10 to 500 mm. The length depends on the intended application of the stent. For instance in peripheral arteries the stent may have a length for instance, in the range 40 to 300 mm. For coronary arteries, the length may be in the range 10 to 50 mm. The diameter may be in the range 2 to 4.5 mm.

For most of the passageways, the diameter of the stents in the first radially expanded conformation is substantially constant along the length of the stent. The stent may flare or have a reduced diameter towards the end portion, in some instances. However, for an insertion into some body passages it may be preferred for the diameter, that is the cross-sectional area, to vary along the length of the stent. For instance it may reduce migration of a device by providing it with a varying diameter along its length such that increased diameter sections and/or reduced diameter sections locate at and interact with, respectively, increased diameter body passageways (for instance openings into a higher volume organ) or reduced diameter sections, for instance at a sphincter. Such varying diameter portions may be provided by use of an appropriate braiding mandrel, or alternatively by a postbraiding heat treatment, changing braid angle during manufacture, or by provision of shaped restraining means such as non-helical filaments etc. Alternatively two or more stent segments maybe fitted together for instance by welding two independently formed sections having the desired shape. One particular application of a varying diameter stent is a stent for use in urological passageways, for instance to overcome benign prostatic hyperplasia.

The filaments from which the braided stents are made are formed of a metal, for instance a surgical steel, and is usually of a type having good elastic properties, for instance a high cobalt stainless steel or an alloy such an Elgiloy. These such materials give a stent having good self-expanding capability.

In addition to the self-expanding capability of the stent, it may be provided with a temperature dependent mechanical characteristic which allows a mechanical property of the stent to be changed by heating the stent from a temperature below transition temperature to above transition temperature. Thus some or all of the filaments may be formed from a shape memory alloy material such as nitinol. In such cases, in the stent prior to implantation, the stent is at a temperature below the transition temperature at which the metal changes from the martensitic structure to the austenitic structure. The filaments are adapted such that a transition from below the transition temperature to above the transition temperature will result in the stent either adopting a radially further expanded configuration or, preferably, retaining the same shape but having an increased resistance to radial collapsing under inwardly exerted pressure.

The stent could also be included in a graft used to replace damaged blood vessels (aneurisms). For instance a stent according to the invention could be surrounded by a sleeve, of a porous or non-porous, elastic or inelastic, material. In this case, the sleeve may be configured so that it is able to deliver a drug to the tissue surrounding the stent when in use. Alternatively a sleeve could include one stent at each end,—secured for instance by suturing or other means, to the stent. The stent can be sterilized before use using standard techniques.

The present invention is illustrated further in the accompanying figures in which:

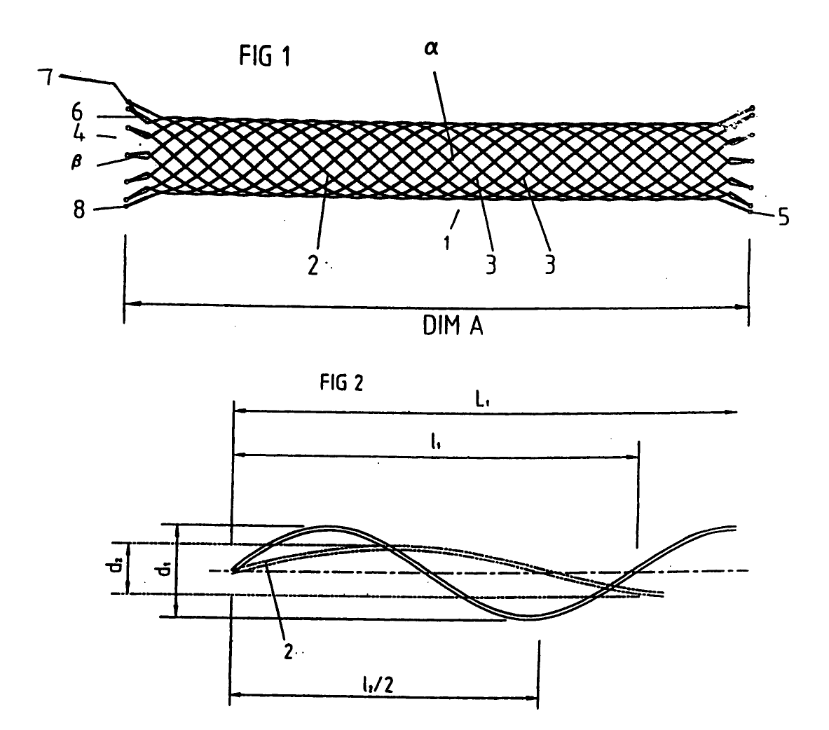

FIG. 1 is a side view of a stent according to the present invention in relaxed, radially expanded condition;

FIG. 2 shows the minimum path of one filament in the stent of a first aspect of the invention;

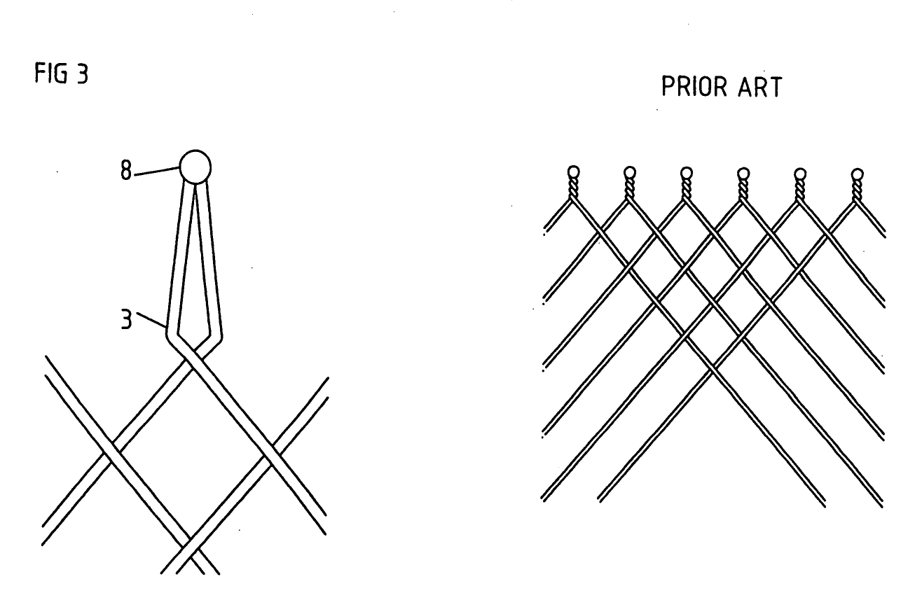

FIG. 3 shows a view of a filament join in an example of the present invention, together with a prior art joining arrangement;

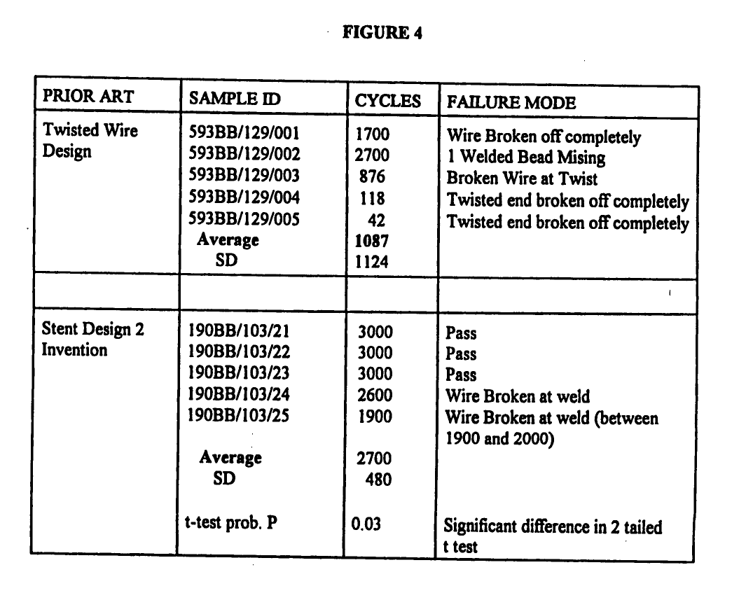

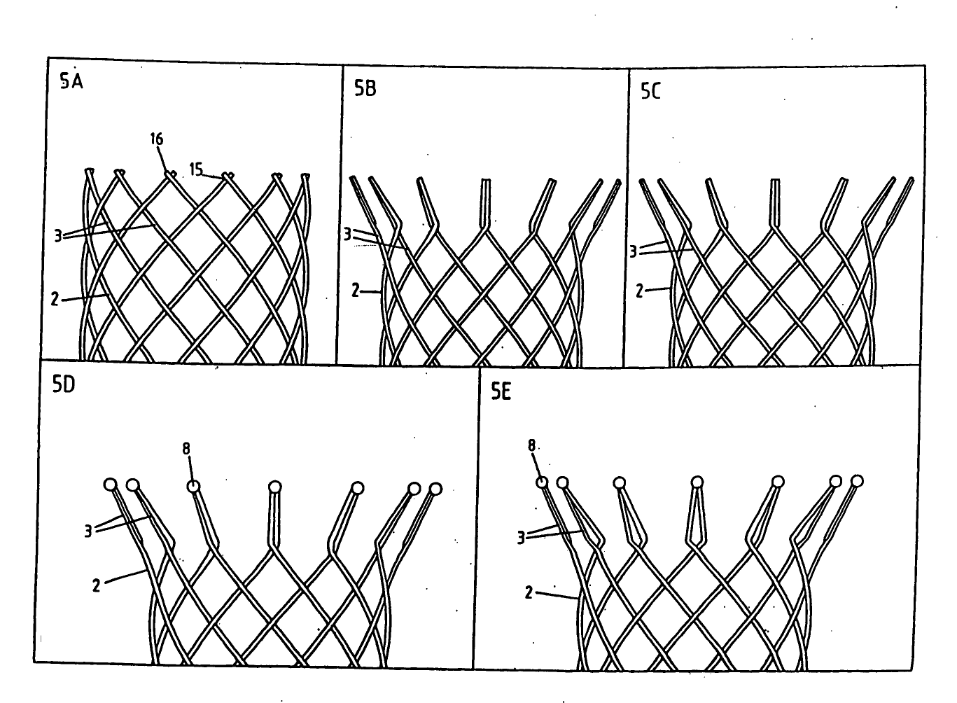

FIG. 4 is data showing the particular benefits of the invention as opposed to an alternative technique;

FIG. 5 is a diagram showing a stent according to the invention during its construction; and

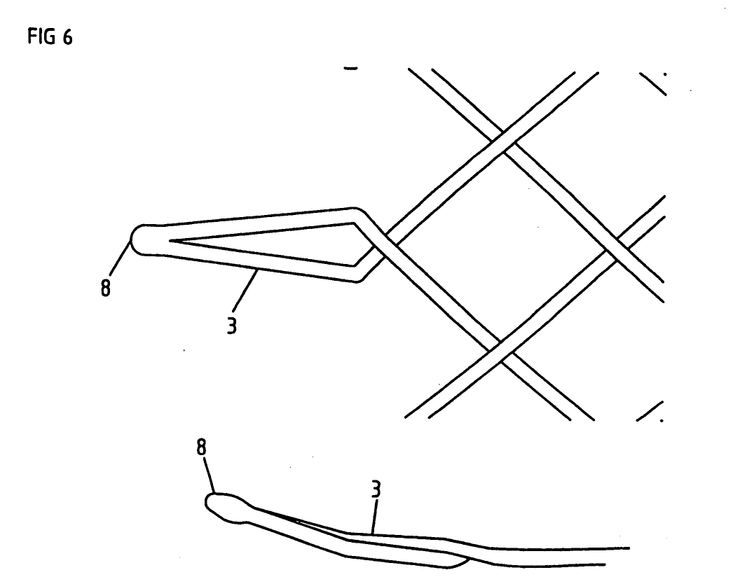

FIG. 6 shows a view of a further example filament join possible in a stent according to the present invention.

As shown in FIG. 1, a stent 1 is formed of twelve wire filaments 2 arranged in right handed helices and twelve filaments 3 arranged in left handed helices. The filaments are braided in a one over/one under pattern. The angle a between the filaments in the radially expanded (relaxed, unloaded) condition is generally in the range 60-90°, in this example in the range 80-90°. Each filament, as shown more clearly in FIG. 2 which is enlarged relative to FIG. 1, executes just over one complete turn (about 1¼ turns) within the length L of the stent. Each turn of the helix has a pitch of 1₁. The diameter of the stent, and of each helix is d₁. In the radially compressed condition and axially extended condition, the length L increases to L₂, whilst the pitch of each helix increases from 1₁ to 1₂ and the diameter reduces from d₁ to d₂. The dotted line in FIG. 2 shows a portion of the filament 2 in its radially compressed state and indicates the length of one half of a turn of the helix as 1₂/2.

Reverting to FIG. 1, at the ends 4 and 5 of the stent a pair of counter-rotating helices are connected together by overlapping them and laying them substantially parallel to one another and forming a bead of metal 8 formed by welding or fusing the wires 6 and 7. The angle β on the tangential plane on the surface of the body between the filaments 6 and 7 is, in this embodiment, about 10°±5°. With the angle β selected as illustrated, in the fully unloaded condition, the ends of the stent do not flare to a disadvantageous degree.

The stent illustrated in FIG. 1 is, for instance, suitable for implanting in a coronary artery. The diameter d1 is in the range 2.5-4.0 mm. The diameter d₂ of the stent, in its axially compressed condition is generally at least V₃ less than diameter d₁, and for instance in the range 0.5 to 2.0 mm. The wire used to form the filaments hews circular section and a diameter of 0.09 mm. The wire is formed from a high cobalt stainless steel or alloy such as Elgiloy. The beads 8 include no filler material but consist only of the material from which the wire of the filaments is formed. The beads generally have a diameter in the range 0.18 to 0.22 mm. When visualized using X-rays, the end portions of the stent including the beads 8 have an increased radiopacity compared to the body of the stent.

The length of the stent in this condition is L₂ (not shown), whilst its diameter is d₂. The angle α₂ between the filaments is reduced by 10 to 60% of the original angle. The stent can be retained in this condition either by exerting radial inwardly directed forces from the stent along its length, or by exerting axially outwardly directed forces at the ends of the stent. The fixing of the ends of the filaments according to the present invention render this latter means of retaining the stent in its radially compressed condition more convenient since it can be achieved by extending pins or other means between the filaments adjacent to the bead 8, or beyond the first crossover points along the length of the stent, at each end and increasing the separation between the ends to extend to the stent in the axial direction. Furthermore, the stent is easier to load into a delivery device.

As well as making it convenient to extend the stent, and stabilize it against flaring at the ends, the joining of the ends of the filaments allows the stent further to be axially compressed by exerting axially inwardly directed pressure against each end, so as to expand the radius of the stent, especially in its central portion, beyond the diameter d₁. The stent can thus be used to exert radially outwardly forces at a greater radial distance from the axis (than d₁) inside the blood vessel, for instance adding to or replacing the step of balloon dilatation prior to stent deployment.

FIGS. 3 and 6 show two alternative joints that may be employed in the present invention. Referring first to FIG. 3, in this example the filaments 3 are joined with a weld which forms a bead 8 and are splayed slightly with a constant angle. Referring to FIG. 6, in this example the join 8 is also formed by a weld, but no bead is formed.

As can be seen from FIG. 6, the joins 8 extend outward radially from the main body of the stent 1, and the filaments 3 are shaped so that the angle at which the join 8 bends outward increases (preferably by 10 to 15°) as the filaments extend towards the join 8.

It has been shown that the particular overlap and alignment configuration of the join has, surprisingly, particular benefits, in terms of strength and flexibility, over other arrangements, such as a simple twisting arrangement. Data to this effect is shown in FIG. 4, which compares the prior art twist design 2 with an example of the invention.

Without the joining of the filament ends such a test might be completely impossible and, even if it were not, the stent ends would be damaged during such an operation. With the angle a being less than 90°, the use of the stent as a dilation device is convenient since a relatively large increase in diameter can be achieved with a relatively small axial reduction in length (as compared to a stent with a higher value of α).

The manufacture of the stent will now be described with reference to FIGS. 5A to 5E. This example differs slightly from that shown in FIG. 3, as the filaments have a differing cross-over configuration near their join.

Firstly, filaments 2, 3 are braided together around a mandrel (not shown) to produce a generally tubular structure. The filaments 2, 3 are wound to satisfy the braid angle requirements discussed above, and the number of filaments selected dependent upon the overall diameter of the stent that is required, as well as the particular application in which the stent is to be used.

Once secured, the filaments 2, 3 are severed around the circumference at position 16, which is located adjacent a series of crossover points. With the filaments secured at 15 and, though not shown, at the other, leading end of the stent portion 17, the stent can be removed from the forming mandrel and heat treated and/or coated as required.

As part of the heat treatment, or even prior to or after heat treatment and coating the ends of some or all of the next-but-one neighboring filaments are bent and aligned parallel to one another in a manner shown in FIG. 5B. Also as part of this process the orientation of the cross-over point adjacent to the ends has its orientation changed in the manner shown in FIG. 5C. Some or all of the aligned ends are then welded together. The weld maybe such that beads 8 are formed, although beads 8 do not need to be formed on each end.

After this step, the stent can be cleaned and coated with a solution of a 1:2 (mole) copolymer of (methacryloyloxy ethyl)-2-(trimethylammonium ethyl)phosphate inner salt with lauryl methacrylate in ethanol (as described in example 2 of WO-A-93/01221) for example.

What is claimed is:

1. A radially self-expanding stent for implantation in a body passage comprises first and second sets of mutually counter-rotating metallic filaments which are braided together and define a tubular stent body having two ends which is mechanically biassed towards a first radially expanded configuration in which it is unconstrained by externally applied forces and can be retained in a second radially compressed configuration, and in which some or all of the filament ends at the ends of the body are fixed together in pairs each consisting of counter-rotating filaments by placing the filaments over one another and placing them adjacent to and substantially parallel to one another and further comprising a join at each end fixing to retain the ends of the filaments in contact with one another, wherein some but not all of the filament ends are welded.

2. A stent according to claim 1, wherein the join generally has a diameter of at least 1.2 times that of the diameter of the filament.

3. A stent according to claim 1, wherein the diameter of the join is no more than 3 times the diameter of the filament.

4. A stent according to claim 1, wherein at least some of the joins provide a shoulder in a rearward axial direction.

5. A stent according to claim 1, wherein the diameter of the join is less than 2.5 times the diameter of the filament.
